(19) United States
(12) Patent Application Publication
(10) Pub. No.: US 2026/0146285 A1
(43) Pub. Date: May 28, 2026

(54) NUCLEIC ACID LOADING REAGENT AND METHOD FOR DETECTING ADSORPTION CAPACITY OF SEQUENCING CHIP USING SAME

(71) Applicant: MGI TECH CO., LTD., Guangdong (CN)

(72) Inventors: Jin YANG, Guangdong (CN); Chongjun XU, Guangdong (CN); Haiyan LUO, Guangdong (CN); Qiong SU, Guangdong (CN)

(21) Appl. No.: 19/122,765

(22) PCT Filed: Oct. 31, 2022
(86) PCT No.: PCT/CN2022/128553

§ 371 Date: Apr. 21, 2025

(51) Int. Cl. C12Q 1/6869

(52) U.S. Cl. CPC C12Q 1/6869

(57) ABSTRACT

The present invention provides a nucleic acid loading reagent and a method for detecting the nucleic acid adsorption capacity of a sequencing chip using same. The method comprises at least one of the following conditions: (i) the nucleic acid loading reagent is at a pH value between 3 and 5; (ii) the nucleic acid loading reagent contains metal ions, the metal ions being, for example, from one or more of the following solutions: MgCl₂, CeCl₃, ZnCl₂, MnCl₂, CaCl₂, CuCl₂, and NiCl₂; and (iii) the chip and the nucleic acid loading reagent are co-incubated for 10 min or above. The present invention provides a quality control scheme for the sequencing chip. The present invention provides a scheme capable of quickly optimizing the loading efficiency and loading bias of a nucleic acid library, and provides a reaction reagent capable of effectively improving the loading efficiency and loading bias of a nucleic acid library.

### TECHNICAL FIELD

The present disclosure relates to the field of biotechnology, in particular to a nucleic acid loading reagent and a method for detecting the adsorption capacity of a sequencing chip using the nucleic acid loading reagent.

### BACKGROUND

With the development of sequencing technology, the increase in societal awareness, and the continuous reduction in sequencing costs, high-throughput gene sequencing has been widely applied in various fields such as scientific research, precision medicine, public health, and public safety. Currently, the cost of whole-genome sequencing (30×, approximately 100Gb of data) based on large-scale parallel sequencing technology is rapidly decreasing from $1000 to $100, which is expected to further reduce the sequencing costs across the entire sequencing market, thereby promoting the broader application of high-throughput sequencing.

There are many factors that determine the quality and cost of high-throughput sequencing, such as biochemical protocols, sequencing materials, or system integration capabilities. One of the key points is whether the sequencing chip used can fully support high-density loading, amplification, and signal detection of the sample to be tested. For large-scale parallel sequencing, the largest diameter of sequencing chip has reached 8 inches, and the loading density of nucleic acid has reached a spacing of 400 nm. Therefore, a single chip can generate more than 50 Gb of effective reads, corresponding to the whole genomes of about 100 individuals, making its scientific and economic value considerable.

However, loading sequencing samples (such as clusters of Illumina or DNA nanoballs (DNB) of MGI) onto sequencing chips still faces many challenges. Mainstream high-throughput sequencers on the market, such as NovaSeq-6000 and DNBSEQ-T7, use sequencing chips prepared by semiconductor processes, which bind to the nucleic acid samples to be tested through active sites. The chip of former sequencer conjugates probes at the active sites, and the bridge PCR amplification starts after nucleic acid samples to be tested binding to the probes. The chip of latter sequencer does not require amplification at the active sites; the nucleic acid samples to be tested undergo rolling circle amplification (RCA) in a test tube and are then adsorbed and loaded onto the chip through interactions such as proteins, surface charges, or ions.

Therefore, it is urgent to provide a solution that can quickly, simply, and accurately control the quality of the sequencing chip, improve the loading efficiency, bias, and robustness of the sequencing library, so as to effectively further reduce the high-throughput sequencing cost, increase the data output, and improve the sequencing quality.

### CONTENT OF THE PRESENT INVENTION

In order to address the deficiencies such as low loading efficiency, high cost and the complexity of quality control in sequencing in the prior art, the present disclosure provides a nucleic acid loading reagent and a method for detecting the adsorption capacity of a sequencing chip using the nucleic acid loading reagent.

A first aspect of the present disclosure provides a method for detecting the nucleic acid adsorption capacity of a sequencing chip, comprising at least one of the following conditions:
(i) a nucleic acid loading reagent has a pH value between 3 and 5;
(ii) the nucleic acid loading reagent contains metal ions which are selected from, for example, those in one or more of the following solutions: MgCl₂, CeCl₃, ZnCl₂, MnCl₂, CaCl₂, CuCl₂, and NiCl₂;
(iii) the chip and the nucleic acid loading reagent are co-incubated for 10 min or more, preferably 15 to 120 min.

In some embodiments, in (i), the pH value is 3 or the pH value is 5.

In some embodiments, in (ii), the metal ions are selected from those in ZnCl₂ or CaCl₂ solution.

In some embodiments, the ZnCl₂ and CaCl₂ solutions have a final concentration between 15 mM and 50 mM, for example, the final concentration of ZnCl₂ solution is 15 mM, 20 mM and 40 mM, preferably 15 mM; the final concentration of CaCl₂ solution is 20 mM and 30 mM, preferably 30 mM.

In some embodiments, the nucleic acid loading reagent is diluted with a buffer, and the buffer is, for example, one or more of a HEPES buffer, a citrate buffer, or a MES buffer.

In some embodiments, the buffer has a pH of 3 to 7.4.

In some embodiments, the buffer is a citrate buffer, and the citrate, for example, has a concentration of 20 mM.

In some embodiments, in (iii), the incubation has a duration of ≥15 min.

A second aspect of the present disclosure provides a nucleic acid loading reagent, which comprises the buffer and the metal ions defined in the method according to the first aspect of the present disclosure.

A third aspect of the present disclosure provides a use of the nucleic acid loading reagent according to the second aspect of the present disclosure in sequencing or preparation of a sequencing reagent.

A fourth aspect of the present disclosure provides a method for quickly detecting the nucleic acid adsorption capacity of a chip, comprising the following steps:
S1: aspirating the nucleic acid loading reagent described in the second aspect of the present disclosure and added with a DNA to be tested with a label, then dropwise loading the nucleic acid loading reagent onto the surface of a sequencing chip or a small chip formed by cutting the same, followed by incubating;
S2: aspirating the supernatant of the nucleic acid loading reagent of S1 to a detection container for detection;
S3: calculating an adsorption ratio.

In some embodiments, in S1, the DNA to be detected is an oligonucleotide single strand.

In some embodiments, the label is a fluorescent label, preferably Cy3.

In some embodiments, the detection container is an ELISA plate or a 0.5 mL transparent centrifuge tube; and the detection is performed in a microplate reader or a Qubit fluorometer.

In some embodiments, the small chip has a size of 8 mm×8 mm or 10 mm×10 mm.

In some embodiments, the incubation has a duration of 15 to 120 min, for example, 15 min.

In some embodiments, the oligonucleotide single strand has a final concentration of 5 to 40 nM, for example, 5 nM.

In some embodiments, the sequences of oligonucleotide single strand are one or more of Poly-nC, Poly-nG, Poly-nT, or Poly-nA, and n is a natural number greater than or equal to 3, for example, 5.

In some embodiments, S2 further comprises adding a control, and the control comprises a negative control and a positive control.

In some embodiments, the negative control is a buffer, for example, a HEPES buffer, a MES buffer, or a citrate buffer.

In some embodiments, the positive control is a DNA to be tested with a label that is not incubated on the sequencing chip or the small chip.

In some embodiments, in S3, the calculation formula for the adsorption ratio is 1−(X−Y)/(Z−Y).

The technical solution of the present disclosure has the following beneficial effects:
(1) providing a quality control scheme for the sequencing chip, which is not restricted to platforms, and is simple to operate, low in cost, and fast;
(2) providing a scheme capable of quickly optimizing the loading efficiency and loading bias of a nucleic acid library;
(3) providing a reaction reagent capable of effectively improving the loading efficiency and loading bias of a nucleic acid library.

### BRIEF DESCRIPTION OF THE DRAWINGS

FIG. 1 is a schematic diagram of sequencing base separation.

FIG. 2 is a schematic diagram of loading a DNB onto a chip.

FIG. 3 is a schematic diagram of sequencing chip adsorption of Oligonucleotide for testing.

FIG. 4 shows chips obtained by cutting in batch 1 (left) and batch 2 (right).

FIG. 5 is a schematic diagram showing the effect of chip batch and adsorption time on DNA adsorption.

FIG. 6 is a schematic diagram of testing the adsorption of Oligonucleotide by a sequencing chip in different pH solutions.

FIG. 7 is a schematic diagram of testing the effect of metal ions on the DNA adsorption by a sequencing chip.

FIG. 8 is a schematic diagram of the effect of metal ions on the DNA adsorption by a sequencing chip.

### DETAILED DESCRIPTION OF TGE PREFERRED EMBODIMENT

The present disclosure will be described in more detail below with reference to the examples and the accompanying drawings. In the present disclosure, all equipment, raw materials, and the like are commercially available or commonly used in the art. Unless otherwise specified, the methods used in the examples are common technologies in the art.

TABLE 1
Reagents and Consumables

| | | Catalog | |
|---|---|---|---|
| Name | Company | Number | Origin |
| 3′ Fluorescently labeled | Sangon | None | China |
| Oligonucleotide | Biotech | | |
| (AAAAA-Cy3) | (Shanghai) | | |
| Citrate buffer (pH 3-6) | Homemade | None | China |
| 1M HEPES buffer | Sangon | E607018-0100 | China |
| | Biotech | | |
| | (Shanghai) | | |
| 0.1M MES buffer | Carnoss | sj1074 | China |
| Ultra-pure water | MGI | None | China |
| Sequencing chip - cut to 8 mm × | MGI | None | China |
| 8 mm | | | |
| 96-well Microplate | Greiner | 655801 | Austria |
| | Bio-One | | |
| 1.5 mL Centrifuge tube | Sangon | F601620 | China |
| | Biotech | | |
| | (Shanghai) | | |
| Tweezers | None | None | China |

TABLE 2
Instruments

| | | Catalog | |
|---|---|---|---|
| Name | Company | Number | Origin |
| Manual single-channel pipette | Eppendorf | None | Germany |
| (Set) | | | |
| Microplate reader | BMG LABTECH | FLUOstar | Germany |
| | | Omega | |

The following is the operational procedure:
1. A DNA oligonucleotide with a fluorescent label was obtained, where the fluorescent label is Cy3 fluorescent dye. The DNA oligonucleotide was single-stranded and may be a poly-oligonucleotide (Poly AAAAA was used in this scheme) or a random combination of four bases ACGT, with a preferred length of ≤30 nt;
2. The sequencing chip, which had been normally prepared (or its scrap material with photolithographic patterns and chemical vapor deposition treatment), was cut into approximately 8-10 mm small square sequencing chips (small sequencing chips for detection) using laser stealth dicing or other methods (e.g., glass knife cutting), and was stored with the front side facing up in a nitrogen cabinet;
3. The working solution of single-stranded DNA oligonucleotides with fluorescent labels was prepared at a final concentration of 5-10 nM. The diluent could be selected from buffers with different pH (3-7.4) and different salt concentrations and types (NaCl, MgCl₂, CaCl₂), etc.) according to needs, so as to study protocols for improving DNB adsorption efficiency, adsorption preference, etc.;
4. The small chip was clamped onto a flat surface with tweezers, with the front side facing up;
5. Approximately 180 μL of the fluorescent dye solution was aspirated with a 200 μL pipette and carefully dropped onto the surface of the small chip, avoiding the liquid from dripping to other places;
6. After reacting (incubating) at room temperature for 15 minutes to 2 hours, approximately 150 μL of the fluorescent dye solution was aspirated from the supernatant using a 200 μL pipette and transferred to a microplate (e.g., Greiner microplate, catalog number: 655801; or a transparent centrifuge tube, e.g., in 0.5 mL specification). Negative controls (buffer only, no fluorescent dye) and positive controls (not incubated on the small chip) were added;
7. If needed, gradient diluted standard curve solutions that were not incubated were prepared and added to the microplate;
8. The microplate reader was turned on, and the corresponding detection wavelengths (530±20 nm for excitation wavelength and 580±30 nm for emission wavelength for Cy3) and the microplate were selected. The corresponding wells were selected, and the Gain was set to a suitable value (e.g., 2000). Tests were performed and repeated three times;
9. The data from three tests were averaged, with the negative control as the background. Taking the positive control as 100%, the adsorption ratio of each condition signal was calculated using the formula: Adsorption Ratio=1−(test−negative control)/(positive control−negative control). The negative control could also be set on the microplate reader, in which case the negative control value did not need to be subtracted. If there was a standard curve, the specific number of adsorbed molar molecules could be calculated;
10. For sequencing chip QC, buffer conditions may be fixed, and multiple batches of chips (different wafers, different surface treatments, etc.) were tested at the same time and compared horizontally. The sequencing chip batch with a higher adsorption ratio should have a stronger ability to load DNB, and the sequencing chip batch with a lower adsorption ratio should have a weaker ability to load DNB;
11. The actual on-machine performance data (too strong leading to over-adsorption, too weak leading to less than 100% DNB loading) need to be comprehensively considered to ultimately establish suitable QC and DNB loading plans.

### Example 1 Testing for Oligo Adsorption on Sequencing Chip

### 1) Preparation of Reaction Solution:

1. The 3′ fluorescently labeled Oligo (using AAAAA-Cy3 (abbreviated as Poly 5A-Cy3) as an example, but not limited to this sequence) was diluted to 100 μM with ultra-pure water and stored at −20° C. for future use;
2. A small amount of the 100 μM Poly 5A-Cy3 was diluted to 1 μM with ultra-pure water and stored at 4° C. for future use.
3. The 1M HEPES buffer (pH=7.4) was diluted to 20 mM with ultra-pure water and stored for future use;
4. Before use, 1 μM Poly 5A-Cy3 was diluted with 20 mM HEPES to a final concentration of 5 nM (e.g., 5 μL Poly 5A-Cy3 was added to 995 μL 20 mM HEPES buffer) to prepare the Poly 5A-Cy3 dye solution;
5. A clean Greiner Bio-One UV microplate was prepared for future use.

### 2) Adsorption Experiment:

1. The small chips (8 mm×8 mm) were clamped onto a clean tabletop and arranged in sequence using tweezers;
2. 180 μL of 5 nM Poly 5A-Cy3 dye solution was carefully aspirated and dropped onto the surface of the small chips, ensuring that no droplets fell off (see FIG. 33);
3. After incubating at room temperature for 15 minutes, 150 μL of the dye solution was aspirated from each small chip using a 200 μL pipette and transferred to designated wells of the microplate;
4. 150 μL of 20 mM HEPES buffer and 5 nM Poly 5A-Cy3 dye solution (without any treatment) were added to the designated wells of the microplate as controls.

### 3) Signal Detection and Analysis

1. The BMG microplate reader was turned on, and the FL endpoint mode was selected. Cy3 dye was selected in the program, with a Gain value selected as 2000, and the corresponding layout was selected;
2. The detection button was clicked, and the process was repeated three times;
3. The values from the three detections were copied into an Excel sheet;
4. The adsorption ratio was calculated as follows:
a) Let the absorbance value under certain conditions be X, the absorbance value of 20 mM HEPES buffer be Y, and the absorbance value of 5 nM Poly 5A-Cy3 be Z;

$$Adsorption Ratio = 1 - ( X - Y ) / ( Z - Y ) ; \qquad b )$$

5. It can be calculated that, after incubating at room temperature for 15 minutes in 20 mM HEPES buffer, the adsorption ratio of the currently treated small chip was 53.67%, demonstrating that this method can be used to study the DNA adsorption on small chips. The results are shown in Table 3.

TABLE 3
Test for Oligo Adsorption on Small Chips (Gain 2000)

| | Fluorescent | Fluorescent | Fluorescent | Adsorption |
|---|---|---|---|---|
| Reaction | Signal - | Signal - | Signal - | Ratio |
| condition | Test 1 | Test 2 | Test 3 | (Average) |
| 20 mM HEPES | 1251 | 1068 | 982 | |
| buffer | | | | |
| Supernatant | 32455 | 30881 | 29975 | 54.52% |
| after Poly | | | | |
| 5A-Cy3 | | | | |
| Adsorption | | | | |
| Supernatant | 31687 | 31004 | 30872 | 54.39% |
| after Poly | | | | |
| 5A-Cy3 | | | | |
| Adsorption | | | | |
| Supernatant | 33244 | 32653 | 32217 | 52.09% |
| after Poly | | | | |
| 5A-Cy3 | | | | |
| Adsorption | | | | |
| 5 nM Poly | 68766 | 66943 | 65497 | |
| 5A-Cy3 Dye | | | | |
| solution | | | | |
| Poly 5A-Cy3 | | | | 53.67% |
| Adsorption | | | | |
| Ratio | | | | |
| (Average of | | | | |
| Triplicate) | | | | |

### Example 2 Sequencing Chip Quality Control and On-machine Verification

### 1) Preparation of Reaction Solution:

1. 1 M MES buffer was used to prepare 20 mM IVIS buffer, pH=4;
2. 20 mM IVIS buffer was used to prepare 20 nM Poly 5A-Cy3 dye solution, with each portion being 4000 μL;
3. 400 μL of 20 mM IVIS buffer was prepared as a negative control.

### 2) Preparation of Chips for Quality Control

The sequencing chips from two different batches (defined as Batch 1 and Batch 2) were cut into sizes of 10 mm×10 mm using laser stealth dicing (FIG. 4).

### 3) Adsorption Experiment:

1. The small chips were clamped onto a clean tabletop and arranged in sequence using tweezers;
2. 180 μL of 5 nM Poly 5A-Cy3 dye solution was carefully aspirated and dropped onto the surface of the small chips, ensuring that no droplets fell off (see FIG. 33);
3. After incubating at room temperature for 15 minutes to 2 hours, 150 μL of the dye solution was aspirated from each small chip using a 200 μL pipette and transferred to designated wells of the microplate;
4. 150 μL of 20 mM MES buffer and 20 nM Poly 5A-Cy3 dye solution (without any treatment on these reagents) were added to the designated wells of the microplate as controls.

### 4) Signal Detection and Analysis

1. The BMG microplate reader was turned on, and the FL endpoint mode was selected. Cy3 dye was selected in the program, with a Gain value selected as 2000;
2. The detection button was clicked, and the process was repeated three times;
3. The values from the three detections were copied into an Excel sheet;
4. The adsorption ratio was calculated as follows:
a) The absorbance value of the supernatant solution (after Poly 5A-Cy3 adsorption) under different pH conditions was set to X. The buffer (without dye solution) was set as a blank control, so there was no need for calculation, and the absorbance value of the non-adsorbed solution was recorded as Z;

$$Adsorption r atio = 1 - X / Z ; \qquad b )$$

5. It can be seen from the results that compared to Batch 1, Batch 2 has higher adsorption amounts at each time point ≥15 minutes, as shown in FIG. 55.

### 5) On-Machine Verification

1. MGISEQ-2000 sequencing chips were prepared using the chips from these two batches. For differentiation, the chips prepared from Batch 1 in the adsorption experiment were named Batch 1-1, and the chips prepared from Batch 2 were named Batch 2-1;
2. DNB preparation, loading, and PE100 sequencing were performed using the MGISEQ-2000 sequencer following the standard sequencing procedure (refer to “MGISEQ-2000RS High-Throughput Sequencing Reagent Kit User Manual”);
3. After comparing indicators such as Q30, DNB loading amount, and effective DNB count, it was found that all indicators of Batch 2-1 were significantly better than those of Batch 1-1, as shown in Table 4;
4. The combined results from the adsorption test and on-machine test indicate that the present disclosure can be used for sequencing chip quality control, with advantages such as low cost and short time.

TABLE 4
On-Machine Performance of Sequencing
Chips Batch 1-1 and Batch 2-1

| Condition | Batch 1-1 | Batch 2-1 |
|---|---|---|
| CycleNumber | 20 | 20 |
| Total Reads(M) | 513.73 | 548.73 |
| Q30(%) | 93.06 | 94.26 |
| ESR(%) | 86.66 | 90.21 |
| ReadNum | 513725618 | 548725794 |
| BaseNum | 10274512360 | 10974515880 |

### Example 3 Test for DNA Adsorption on Sequencing Chips at Different pH Values

### [0110]1) Preparation of Reaction Solution:

1. Citrate buffers with different pH values (pH 3-6) were prepared, with a sodium citrate concentration of 20 mM; two other buffers were prepared at the same time: 20 mM MES buffer at pH 4, and 20 mM HEPES buffer at pH 7.4;
2. 5 nM Poly 5A-Cy3 dye solutions were prepared using citrate buffers at different pH values, with each portion being 400 μL;
3. Citrate buffers without and with fluorescence (Poly 5A-Cy3) at different pH values were prepared as negative and positive controls.

### 2) Adsorption Experiment:

1. The small chips (8 mm×8 mm) were clamped onto a clean tabletop and arranged in sequence using tweezers;
2. 180 μL of 5 nM Poly 5A-Cy3 dye solution was carefully aspirated and dropped onto the surface of the small chips, ensuring that no droplets fell off (see FIG. 6);
3. After incubating at room temperature for 15 minutes, 150 μL of the dye solution was aspirated from each small chip using a 200 μL pipette and transferred to designated wells of the microplate;
4. Negative control (20 mM HEPES buffer without fluorescence) was added to the designated wells of the microplate; and positive controls (20 mM HEPES buffer containing 5 nM Poly 5A-Cy3, 20 mM MES buffer containing 5 nM Poly 5A-Cy3, and citrate buffers at pH 3-6 containing 5 nM Poly 5A-Cy3) were added, respectively. None of these reagents were treated with any adsorption.

### 3) Signal Detection and Analysis

1. The BMG microplate reader was turned on, and the FL endpoint mode was selected. Cy3 dye was selected in the program, with a Gain value selected as 2000, and the corresponding layout was selected according to Table 3;
2. The detection button was clicked, and the process was repeated three times;
3. The values from the triplicate were copied into an Excel sheet;
4. The adsorption ratio was calculated as follows:

For each pH condition, the absorbance values were processed individually as follows: the absorbance value of the supernatant solution after Poly 5A-Cy3 adsorption was set to X, the absorbance value of the buffer (HEPES without dye) was set to Y, and the absorbance value of the dye solution without absorption was set to Z; adsorption ratio=1−(X−Y)/(Z−Y);
5. It can be calculated that, as the pH decreases, the overall DNA adsorption capacity of the small chips gradually increases, from 36.36% to more than 68%. However, between pH 4.5 and pH 5, the DNA adsorption rate remains essentially unchanged as the pH decreases. This data indicates that for DNA adsorption on small chips, the pH should be appropriately lowered. In practical sequencing, a pH value of less than 4 might cause loading abnormalities, so the focus should be on studying the DNB loading conditions between pH 4 and 4.5. Additionally, MES shows a higher adsorption ratio of DNA Oligo compared to citrate at the same pH, making it more advantageous.

TABLE 5
Adsorption Ratio of Oligo on Sequencing
Chips at Different pH Values (Gain 2000)

| Reaction | | | | |
|---|---|---|---|---|
| conditions - | Fluorescent | Fluorescent | Fluorescent | Adsorption |
| 15 min at room | Signal - | Signal - | Signal - | Ratio |
| temperature | Test 1 | Test 2 | Test 3 | (Average) |
| 20 mM HEPES | 6097 | 5891 | 5760 | — |
| Buffer - | | | | |
| Without Cy3 | | | | |
| pH 7.4 | 50883 | 49566 | 49981 | 36.36% |
| (HEPES) - | | | | |
| Supernatant | | | | |
| after Cy3 | | | | |
| adsorption | | | | |
| pH 6 (Citrate) - | 31955 | 31298 | 30765 | 40.79% |
| Supernatant | | | | |
| after Cy3 | | | | |
| adsorption | | | | |
| pH 5 (Citrate) - | 26135 | 27150 | 26787 | 49.85% |
| Supernatant | | | | |
| after Cy3 | | | | |
| adsorption | | | | |
| pH 4.5 | 26828 | 26871 | 25693 | 48.92% |
| (Citrate) - | | | | |
| Supernatant | | | | |
| after Cy3 | | | | |
| adsorption | | | | |
| pH 4 (Citrate) - | 39519 | 38948 | 38256 | 53.45% |
| Supernatant | | | | |
| after Cy3 | | | | |
| adsorption | | | | |
| pH 4 (MES) - | 24786 | 26510 | 25800 | 66.45% |
| Supernatant | | | | |
| after Cy3 | | | | |
| adsorption | | | | |
| pH 3 (Citrate) - | 15534 | 15344 | 15071 | 68.71% |
| Supernatant | | | | |
| after Cy3 | | | | |
| adsorption | | | | |
| pH 7.4 | 75663 | 74592 | 75981 | |
| (HEPES) - No | | | | |
| Cy3 absorption | | | | |
| pH 6 (Citrate) - | 49343 | 48866 | 48357 | |
| No Cy3 | | | | |
| absorption | | | | |
| pH 5 (Citrate) - | 47712 | 47335 | 46980 | |
| No Cy3 | | | | |
| absorption | | | | |
| pH 4.5 | 46360 | 46092 | 45973 | |
| (Citrate) - | | | | |
| No Cy3 | | | | |
| absorption | | | | |
| pH 4 (Citrate) - | 76890 | 77154 | 76338 | |
| No Cy3 | | | | |
| absorption | | | | |
| pH 4 (MES) - | 64085 | 65713 | 64838 | |
| No Cy3 | | | | |
| absorption | | | | |
| pH 3 (Citrate) - | 36105 | 35887 | 35873 | |
| No Cy3 | | | | |
| absorption | | | | |

### Example 4 Adsorption Preference and Adsorption Efficiency of the Sequencing Chip for DNA

For the present disclosure, due to the advantages mentioned above, the present disclosure provides a good experimental platform for optimizing the efficiency and adsorption preference of the sequencing chip for DNA samples. Therefore, the following experiments were designed:

### 1. The Adsorption of Four Bases a, C, G and T on the Surface of the Sequencer Chip was Tested.

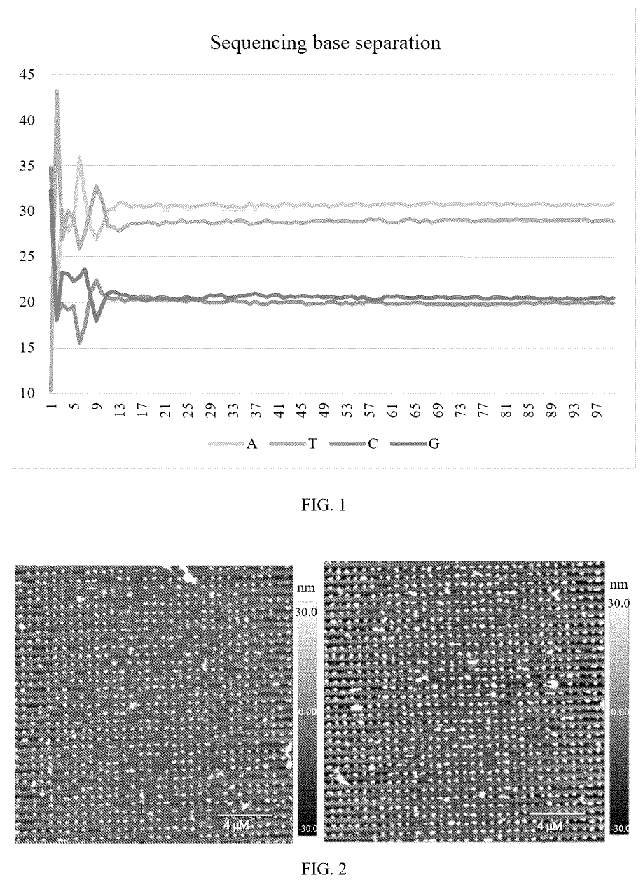

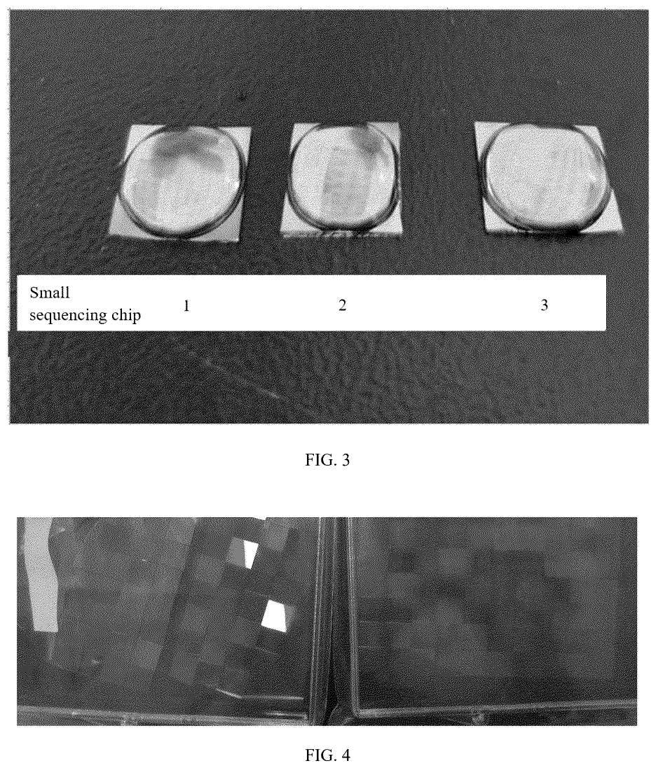

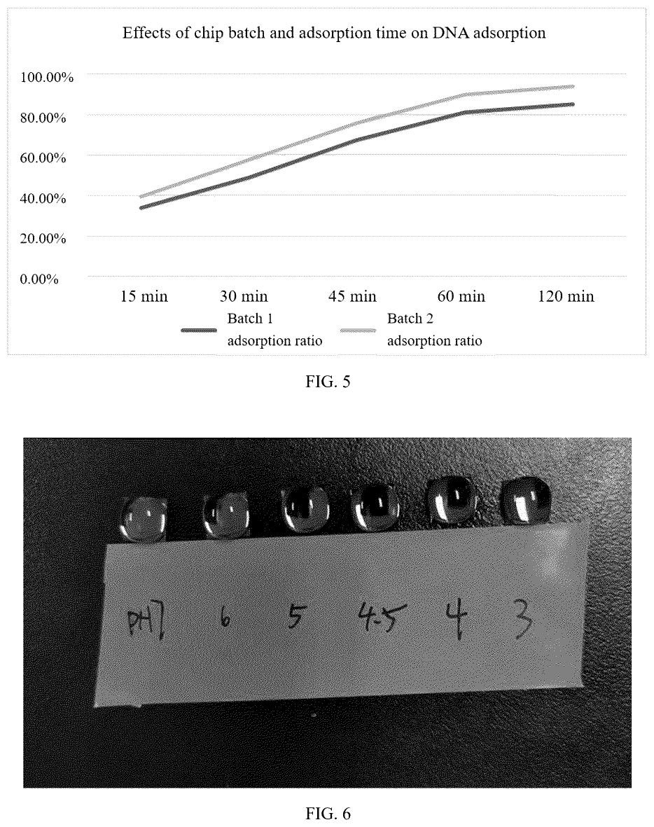

A (adenine deoxyribonucleotide), C (cytosine deoxyribonucleotide), G (guanine deoxyribonucleotide), and T (thymine deoxyribonucleotide) are components of DNA, where A pairs with T, and C pairs with G. Considering the randomness of DNA extraction and library preparation, if DNB loading is assumed to be sufficiently random, then in the sequences obtained by sequencing, it should be A=T, and C=G. However, in actual sequencing, due to various reasons, A and T, and C and G are often unequal. One of the reasons is the selective preference of the chip for different bases during DNB loading (FIG. 1). To investigate this, Poly-5A-Cy3, Poly-5C-Cy3, Poly-5G-Cy3, and Poly-5T-Cy3 were used, following the methods in Example 1, to verify the adsorption on the sequencing chip. The results show that all four bases can be adsorbed and detected, among which Poly-5C-Cy3 not only has a high signal, but also has a fast adsorption rate. It can also be seen that the adsorption rates of different bases on the chip are inconsistent, possibly related to binding sites (pyrimidines have smaller binding sites than purines), as shown in Table 6.

TABLE 6
Adsorption of Four Bases, ACGT, on the Surface
of the Sequencing Chip (Gain 2000)

| | Treatment Condition - 20 mM | Supernatant | |
|---|---|---|---|
| | HEPES solution, room | Fluorescent | Adsorption |
| Base Type | temperature for 10 min | Signal | Ratio |
| Poly-5A-Cy3 | Sequencing chip adsorption | 91714 | 25.01% |
| | No sequencing chip | 122306 | |
| | adsorption | | |
| Poly-5C-Cy3 | Sequencing chip adsorption | 64815 | 50.44% |
| | No sequencing chip | 130773 | |
| | adsorption | | |
| Poly-5G-Cy3 | Sequencing chip adsorption | 38581 | 33.94% |
| | No sequencing chip | 58403 | |
| | adsorption | | |
| Poly-5T-Cy3 | Sequencing chip adsorption | 47995 | 50.59% |
| | No sequencing chip | 97136 | |
| | adsorption | | |

### 2. Testing the Effect of Metal Ions on DNA Adsorption and On-Machine Verification

Metal ions carry positive charges, while the bases and phosphate groups of DNA generally carry negative charges in solution (e.g., pH 4-8). Therefore, through electrostatic interactions, metal ions can bind to DNA, assist in DNA folding, and possibly promote the interaction between DNA molecules and the surface of sequencing chips through salt bridges or electrostatic shielding. In DNBSEQ technology, the DNB loading system contains Na⁺ and Mg2⁺ metal ions, but the issue of low DNB loading efficiency still frequently occurs, which affects data output. As shown in FIG. 2, the left image shows low loading efficiency, resulting in many empty spots; and the right image shows high loading efficiency, with almost no empty spots.

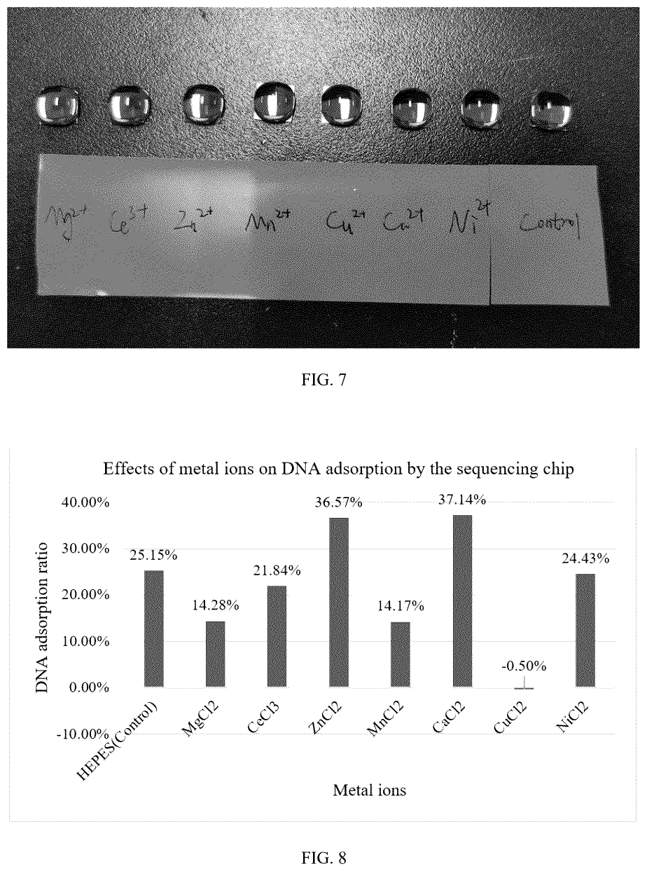

In the present disclosure, six divalent metal ions and one trivalent metal ion were tested, including five transition metal ions, as shown in Table 7. The metal ions were prepared into a 50 μM solution using 20 mM HEPES at pH 7.4, which was then used to prepare 5 nM Poly-5A-Cy3. The adsorption verification was conducted on the sequencing chip, as shown in FIG. 7.

Experimental data indicate that ZnCl₂ and CaCl₂ have certain effects in promoting DNA adsorption by the sequencing chip, but the effects of other tested ions are not significant and may have a certain impact on the fluorescence signal, as shown in FIG. 8. This test did not verify the ion concentration gradient, and it is expected that the ion concentration gradient would also have a certain impact on adsorption.

TABLE 7
Effect of Metal Ions on DNA Adsorption by the Sequencing Chip

| Metal | | Buffer | Reaction | |
|---|---|---|---|---|
| Ion | Concentration | Solution | Time | Remarks |
| MgCl₂ | 50 mM | 20 mM HEPES | 10 min | |
| CeCl₃ | 50 mM | 20 mM HEPES | 10 min | Transition Metal |
| ZnCl₂ | 50 mM | 20 mM HEPES | 10 min | Transition Metal |
| MnCl₂ | 50 mM | 20 mM HEPES | 10 min | Transition Metal |
| CaCl₂ | 50 mM | 20 mM HEPES | 10 min | |
| CuCl₂ | 50 mM | 20 mM HEPES | 10 min | Transition Metal |
| NiCl₂ | 50 mM | 20 mM HEPES | 10 min | Transition Metal |

Further, two metal salts, ZnCl₂ and CaCl₂, were tested on the MGISEQ-2000 sequencer, with existing standard conditions as the control group. The test scheme was as follows:
(1) After completing the DNB preparation, ZnCl₂ and CaCl₂ were added to the DNB loading system at a final concentration of 15-40 mM, and DNB was then loaded on the MGISEQ-2000 sequencing chip;
(2) SE20 sequencing was performed on the MGISEQ-2000 sequencer, and indicators such as Q30, effective data volume, and the number of DNBs were analyzed.

The results are shown in Table 8. The results show that both ZnCl₂ and CaCl₂ can increase the number of DNBs, indicating that the efficiency of DNB loading onto the chip is improved. The concentrations of ZnCl₂ and CaCl₂ can range from 15 to 50 mM.

In addition, after the addition of ZnCl₂ and CaCl₂, the bias in DNB loading on the sequencing chip was improved, as reflected by a decrease in the A-T value, which is more in line with the distribution of bases in the library and reference genome. Therefore, this can further improve sequencing quality and customer satisfaction.

TABLE 8
Effect of Metal Ions on Improving DNB Loading Efficiency

| | | 15 mM | 20 mM | 40 mM | 20 mM | 30 mM |
|---|---|---|---|---|---|---|
| Condition | Control | ZnCl₂ | ZnCl₂ | ZnCl₂ | CaCl₂ | CaCl₂ |
| CycleNumber | 20 | 20 | 20 | 20 | 20 | 20 |
| Total Reads(M) | 487.47 | 547.47 | 535.71 | 537.65 | 518.65 | 537.16 |
| Q30(%) | 92.55 | 94.44 | 94.25 | 94.64 | 94.23 | 94.38 |
| ESR(%) | 80.14 | 90 | 89.94 | 90.27 | 87.05 | 90.12 |
| ReadNum | 487468488 | 547470263 | 535706663 | 537648305 | 518647562 | 537159282 |
| BaseNum | 9749369760 | 10949405260 | 10714133260 | 10752966100 | 10372951240 | 10743185640 |
| AT Separation | 1.18 | 0.88 | 0.72 | 0.65 | 0.91 | 0.82 |
| (A-T) | | | | | | |

Although specific embodiments of the present disclosure have been described above, those skilled in the art should understand that these are only examples, and various changes or modifications can be made to these embodiments without departing from the principles and essence of the present disclosure. Therefore, the scope of protection of the present disclosure is defined by the appended claims.

1. A method for detecting the nucleic acid adsorption capacity of a sequencing chip, comprising at least one of the following conditions:
(i) a nucleic acid loading reagent has a pH value between 3 and 5;
(ii) the nucleic acid loading reagent contains metal ions;
(iii) the chip and the nucleic acid loading reagent are co-incubated for 10 minutes of more.

2. The method according to claim 1, wherein the method further comprises at least one of the following conditions:
1) in (i), the pH value is 3 or the pH value is 5;
(2) the metal ions are selected from those in one or more of the following solutions: MgCl₂, CeCl₃, ZnCl₂, MnCl₂, CaCl₂, CuCl₂, and NiCl₂;
(3) the chip and the nucleic acid loading reagent are co-incubated for 15 to 120 minutes.

3. The method according to claim 2, wherein in (ii), the metal ions are selected from those in ZnCl₂ or CaCl₂ solution.

4. The method according to claim 3, wherein the method further comprises at least one of the following conditions:
(1) the ZnCl₂ and CaCl₂ solutions have a final concentration between 15 mM and 50 mM;
(2) the nucleic acid loading reagent is diluted with a buffer.

5. The method according to claim 4, wherein the method further comprises at least one of the following conditions:
(1) the buffer is selected from one or more of a HEPES buffer, a citrate buffer, and a MES buffer;
(2) the final concentration of ZnCl₂ solution is 15 mM, 20 mM and 40 mM; the final concentration of CaCl₂) solution is 20 mM and 30 mM.

6. The method according to claim 5, wherein the method further comprises at least one of the following conditions:
(1) the buffer has a pH of 3 to 7.4;
(2) the buffer is a citrate buffer.

7. The method according to claim 6, wherein the citrate has a concentration of 20 mM.

8. A nucleic acid loading reagent, comprising a buffer and the metal ions defined in the method according to claim 2;
the buffer is selected from one or more of a HEPES buffer, a citrate buffer, and a MES buffer.

9. (canceled)

10. A method for quickly detecting the nucleic acid adsorption capacity of a chip, comprising the following steps:
S1: aspirating the nucleic acid loading reagent described in claim 6 and added with a DNA to be tested with a label, then dropwise loading the nucleic acid loading reagent onto the surface of a sequencing chip or a small chip formed by cutting the same, followed by incubating;
S2: aspirating the supernatant of the nucleic acid loading reagent of S1 to a detection container for detection;
S3: calculating an adsorption ratio.

11. The method according to claim 10, wherein the method comprises at least one of the following conditions:
(1) in S1, the DNA to be detected is an oligonucleotide single strand;
(2) the label is a fluorescent label;
(3) the detection container is an ELISA plate or a 0.5 mL transparent centrifuge tube; and the detection is performed in a microplate reader or a Qubit fluorometer;
(4) the small chip has a size of 8 mm×8 mm or 10 mm×10 mm;
(5) the incubation has a duration of 15 to 120 min.

12. The method according to claim 11, wherein the method further comprises at least one of the following conditions:
(1) the oligonucleotide single strand has a final concentration of 5 to 40 nM;
(2) the oligonucleotide single strand is one or more dNTP of Poly-nC, Poly-nG, Poly-nT, or Poly-nA, and n is a natural number greater than or equal to 3.

13. The method according to claim 10, wherein S2 further comprises adding a control, and the control comprises a negative control and a positive control.

14. The method according to claim 13, wherein the method further comprises at least one of the following conditions:
(1) the negative control is a buffer;
(2) the positive control is a DNA to be tested with a label that is not incubated on the sequencing chip or the small chip.

15. (canceled)

16. The method according to claim 14, wherein the negative control is a buffer selected from a HEPES buffer, a MES buffer, or a citrate buffer.

17. The method according to claim 12, wherein n is 5.

18. The method according to 11, wherein the fluorescent label is Cy3.

19. The nucleic acid loading reagent according to claim 8, wherein the nucleic acid loading reagent is further defined by at least one of the following:
(1) metal ions are selected from those in ZnCl₂ or CaCl₂ solution;
(2) the buffer has a pH of 3 to 7.4;
(3) the buffer is a citrate buffer.

20. The nucleic acid loading reagent according to claim 19, wherein the nucleic acid loading reagent is further defined by at least one of the following:
(1) the ZnCl₂ and CaCl₂ solutions have a final concentration between 15 mM and 50 mM;
(2) the citrate has a concentration of 20 mM.

21. The nucleic acid loading reagent according to claim 20, wherein the final concentration of ZnCl₂ solution is 15 mM, 20 mM and 40 mM; the final concentration of CaCl₂ solution is 20 mM and 30 mM.
